(19) United States
(12) Patent Application Publication
(10) Pub. No.: US 2008/0221547 A1
(43) Pub. Date: Sep. 11, 2008

(54) Medicine Bottle Configuration and Method of Using Same

(76) Inventor: David A. Monty, Fuquay-Varina, NC (US)

Correspondence Address: COATS & BENNETT, PLLC, 1400 Crescent Green, Suite 300, Cary, NC, 27518, US

(21) Appl. No.: 11/742,723

(22) Filed: May 1, 2007

Related U.S. Application Data
(60) Provisional application No. 60/905,376, filed on Mar. 7, 2007.

(51) Int. Cl. A61M 31/00; A61M 19/00; A61M 5/30

(52) U.S. Cl. 604/500; 604/403; 604/68

(57) ABSTRACT

A medicine bottle assembly includes a medicine bottle having a storage cavity and an outlet port, with the outlet port disposed on an exterior of the bottle and in fluid communication with the storage cavity. Liquid medicament is disposed in the storage cavity and defines a current fluid level. The outlet port is advantageously disposed entirely below the current fluid level and oriented generally upward. The outlet port is operative to selectively allow flow of the medicament out of the bottle. The assembly may further include a needless syringe having an angled tip. The exterior of the bottle may include a recess disposed above the outlet port, with the recess adapted to receive and retain the syringe. The recess is advantageously aligned with the outlet port. Other aspects and methods are also disclosed.

This application claims the benefit of U.S. Provisional Application No. 60/905,376, filed 7 Mar. 2007.

### BACKGROUND

The present invention relates to medicine dispensers, and more particularly to devices for extracting liquid medicine from container bottles.

Prior to administering liquid medicine to children, invalids, and the like, it is frequently necessary to first draw the medicine from the container bottle into a syringe. Use of the syringe allows for proper dosing to be more readily achieved, and facilitates administration to patients, like children, who may have trouble with or resist other oral administration techniques. And, for many liquid medicament therapies, administering a precise dosage is essential to success of the treatment.

The syringes employed for such use are typically referred to as needleless syringes. Typically, these syringes have a blunt-tipped nose which is advanced into the bottle. Then, the plunger of the syringe is manipulated to draw a precise amount of liquid into the chamber of the syringe's barrel. The syringe is next withdrawn from the bottle and used to administer the dosage of liquid medicament to the patient.

However, the syringe and bottle combination is not always easy to use. For example, two hands may be needed to draw medicine with the syringe, one to hold the barrel, and one to pull the plunger; this leaves no hand to hold the medicine bottle. Also, if the bottle is full, it is easy to over-insert the syringe, thereby spilling some of the medicament. Conversely, when the level of medicament in the bottle is low, the task of holding the bottle in a tilted orientation while operating the syringe at the same time can prove difficult. Further, it is difficult to see the fluid level in the syringe with the syringe inserted in the bottle.

Thus, there remains a need for alternative approaches to medicine dispensing, advantageously approaches that allow for greater ease of use with medicines that are administered with needleless syringes and stored in container bottles.

### SUMMARY

In some, but not necessarily all embodiments, the present invention provides a medicine bottle assembly that allows the syringe to be easily filled with medicament stored from a container bottle, and for the fluid level in the syringe to be easily viewable during the filling process.

In one illustrative embodiment, a medicine bottle assembly comprises a medicine bottle having an interior storage cavity and an outlet port, the outlet port disposed on an exterior of the bottle and in fluid communication with the storage cavity. Liquid medicament is disposed in the storage cavity and defines a current fluid level. The outlet port is disposed entirely below the current fluid level and oriented generally upward. The outlet port is operative to selectively allow flow of the medicament out of the bottle. A valve may be disposed proximate the outlet port and be operative to control flow of the medicament through the outlet port. The assembly may further include a needless syringe having an angled tip. The valve may be automatically responsive to insertion of a hollow syringe tip thereinto to allow the medicament to flow out the outlet port. The exterior of the bottle may include a recess disposed above the outlet port, with the recess adapted to receive and retain a needless syringe. The recess is advantageously aligned with the outlet port.

In another embodiment, the present invention provides a method of dispensing medicine from a container bottle. The method comprises: inserting a tip of a needless syringe into an outlet port; the outlet port disposed at a level below a current fill level of medicament stored in the bottle and oriented generally vertically upward; moving an amount of medicament from the bottle into the syringe while the syringe is mated to the outlet port; decoupling the syringe from the outlet port; and thereafter, expelling medicament from the syringe. The method may further include retaining the syringe in a recess formed on an exterior of the bottle during the moving of an amount of medicament from the bottle into the syringe. The method may further include disposing the syringe so that graduation markings associated therewith are visible during the moving an amount of medicament from the bottle into the syringe.

In another embodiment, a medicine bottle assembly comprises a medicine bottle having an interior storage cavity and an exterior surface. Liquid medicament is disposed in the storage cavity. A needless syringe is removably coupled to the bottle. The bottle exterior surface defines a recess sized and configured to receive the syringe. The syringe is disposed in the recess and is in operative fluid communication with the medicament in the storage cavity.

Other aspects of various embodiments of the inventive apparatus and related methods are also disclosed in the following description. The various aspects may be used alone or in any combination, as is desired.

### BRIEF DESCRIPTION OF THE DRAWINGS

FIG. 1 shows a perspective view of a medicine bottle assembly according to one embodiment of the present invention, with the syringe mated to the bottle.

FIG. 2 shows a perspective view of the bottle of FIG. 1 with the filling cap removed.

FIG. 3 shows a partial cross-section of the bottle along line III-III of FIG. 2 with the valve in the sealing position.

FIG. 4 shows a bottom view of one embodiment of a flexible membrane.

FIG. 5 shows a partial cross-section of the bottle along line III-III of FIG. 2 with the valve in the dispensing position.

FIG. 6 shows a syringe.

FIG. 7 shows a side view of a medicine bottle assembly according to another embodiment of the present invention, with the syringe being filled.

FIG. 8 shows a perspective view of a bottle suitable for another embodiment of the present invention.

FIG. 9 shows another embodiment of a syringe inserted in another embodiment of a bottle recess.

FIG. 10 shows a view similar to FIG. 3 where the valve employs another embodiment of a flexible membrane.

### DETAILED DESCRIPTION

In some, but not necessarily all embodiments, the present invention provides a medicine bottle assembly that allows the syringe to be easily filled with medicament stored in a container bottle, and for the fluid level in the syringe to be easily viewable during the filling process. The bottle advantageously includes an outlet port that is disposed below the fluid level of the medicament in the bottle so that the medicament is easily provided to the syringe during the syringe filling process. In addition, a recess in the bottle is aligned with the outlet port, and the syringe is releasably captured in the recess, for storage and/or during the syringe filling process. The syringe advantageously includes graduation markings that are visible while the syringe is disposed in the recess. An valve, advantageously an automatic valve, controls flow of medicament through the outlet port so that medicament may flow when the syringe is inserted into the outlet port, but is prevented from doing so when the syringe is absent. The various aspects of the present invention, such as the location of the outlet port, and the presence and/or configuration of the recess, may be used alone or in any combination, as is desired.

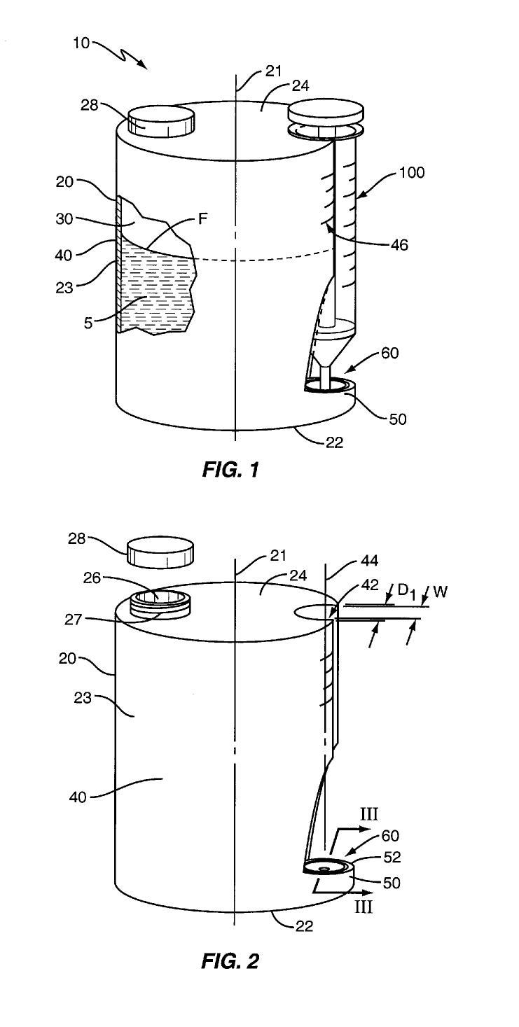

One illustrative embodiment of a medicine bottle assembly according to the invention is shown in FIG. 1, and generally indicated at 10. The medicine bottle assembly 10 includes a bottle 20, a valve 70, and a syringe 100. The bottle 20 includes a bottom 22, a top 24, an intervening sidewall 23, and an interior storage cavity 30. The bottom 22 is typically relatively flat so that the bottle 20 may be stably supported by a suitable tabletop or the like. The top 24 advantageously includes a fill opening 26 that leads to storage cavity 30. Removable cap 28 may mate with bottle 20 to selectively close fill opening 26, such as via external threads 27 on bottle. See FIG. 2. The removable cap 28 may advantageously take the form of a locking type cap that allows entry of air, but militates against liquid escape, of a type known in the art. The main portion of the bottle 20 may be generally cylindrical, but any other conventional medicine bottle shape may be used such as somewhat rectangular, etc. Indeed, the bottle 20 may have a shape suggestive of an animal, cartoon character, toy, or the like, if desired. Bottle exterior surface 40 advantageously includes a recess 42 on sidewall 23 for receiving syringe 100, as discussed further below. The lower portion of bottle 20 includes an outwardly extending flange 50 having an aperture 54 through its top surface 52, forming outlet port 60 as discussed further below. Bottle 20 may optionally include an additional rearwardly extending flange (not shown) for additional stability, if desired. This outlet port 60 is advantageously vertically aligned with recess 42. Interior storage cavity 30 is defined by bottle 20, and provides space for storing medicament 5. A portion of storage cavity 30 extends into flange 50, so that outlet port 60 may be in fluid communication with storage cavity 30. The interior floor of storage cavity 30 may be advantageously sloped or otherwise contoured to encourage fluid flow toward flange 50. The bottle 20 may be made from any suitable material known in the art, including glass and various polymer materials.

The medicament 5 stored in bottle cavity 30 is liquid, and therefore fills bottle cavity 30 to a fluid fill level F that varies based on the amount of medicament 5 in bottle 20. See FIG. 1. Flange 50 is located very low on bottle 20, so that the outlet port 60 associated with flange 50 is disposed below the fluid level F of medicament 5 in bottle 20, except when the bottle 20 is almost empty. Because the outlet port 60 is located below the fluid level F, and bottle 20 cavity 30 is subject to atmospheric pressure, medicament 5 is easily supplied to outlet port 60.

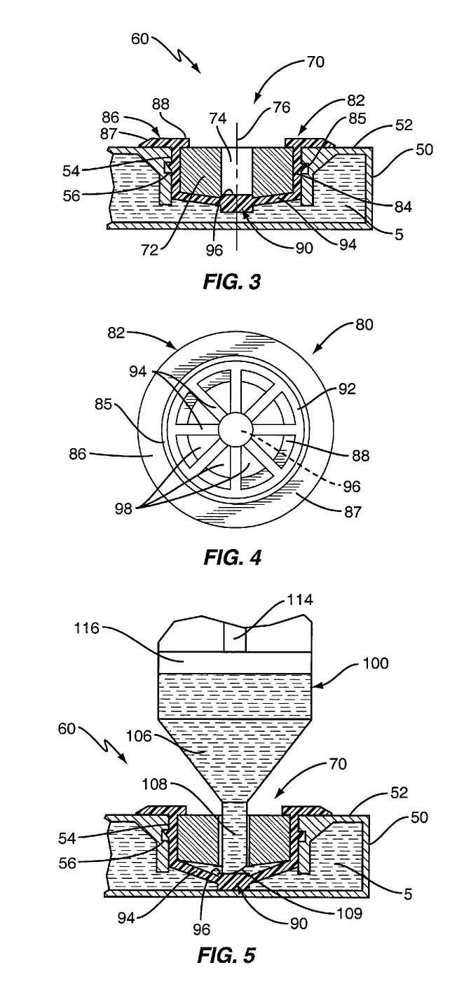

Valve 70 is mounted to bottle 20, and provides a control means for controlling the flow of medicament 5 out of bottle 20 via outlet port 60. Valve 70, in one embodiment, includes an annular insert 72 and a flexible membrane 80. See FIG. 3. Annular insert 72 is sized just smaller than bottle aperture 54 so as to fit therein. Annular insert 72 includes central bore 74 that is shaped and sized to accept nose 108 of syringe 100 in a sliding fit, as discussed below. In some embodiments, the annular insert 72 may be made of a slightly flexible material, and bore 74 may be slightly deformed by the insertion of syringe nose 108, so that a peripheral seal is formed therebetween. The lower face of annular insert 70 may be flat or, more advantageously, is slightly downwardly tapered toward the axis 76 of bore 74. Flexible membrane 80 includes a retaining section 82 and a sealing section 90. Retaining section 82 is generally cylindrical in shape, with a generally “L” or “T” shaped cross-section. For the embodiment of FIG. 3, retaining section 82 includes a vertical wall 84 and a horizontal flange 86 extending from the upper portion of vertical wall 84. Vertical wall 84 may advantageously include an outwardly extending rib 85 for mating with corresponding retaining groove 56 in the wall of aperture 54. This rib 85 may be continuous, or may be formed in discrete sections, such as by a series of barbs. The upper face of rib 85 is advantageously relatively flat and oriented generally transverse to bore axis 76. The lower face of rib 85 is advantageously angled, so as to facilitate the insertion of flexible membrane 80 into bottle aperture 54. Horizontal flange 86 includes an outboard section 87 that overlies a portion of flange upper surface 52 proximate aperture 54, and an inboard section 88 that overlies a portion of annular insert 72. Sealing section 90, in the embodiment of FIGS. 3-5, includes a peripheral rim 92, a plurality of spokes or arms 94 leading to a central hub area 96, and a plurality of intervening spaces or pores 98. See FIG. 4. The peripheral rim 92 joins with, or is formed by, the lower portion of vertical wall 84. Spokes 94 extend inward from rim 92 to central hub 96 and provide flexible support for hub 96. Spokes 94 are spaced from one another so as to form a plurality of intervening spaces or pores 98. It is intended that medicament 5 will flow through these pores 98 during the syringe filling process, as discussed further below. Central hub 96 is a solid area that is slightly larger in size than bore 74 and aligned therewith. In some embodiments, the central hub 96 may include a downwardly extending projection or area, which helps prevent over-extension of the flexible membrane 80 by abutting against the interior wall of bottle 20. See FIG. 5. The flexible membrane 80 should be made from a suitable elastomeric material, such as neoprene, rubber, flexible nylon, or other elastic material suitable for contact with medicament.

Flexible membrane 80 is moveable between a sealing position (FIG. 3) and a dispensing position (FIG. 5). The normal or natural position of flexible membrane 80 is the sealing position. In the sealing position, central hub 96 abuts bore 74 so as to seal off bore 74 from bottle cavity 30. Thus, flexible membrane 80 acts to cut off fluid communication from bottle cavity 30 to bore 74 in the sealing position. It should be noted that the flexible membrane 80 is advantageously slightly distended by the presence of annular insert 72, so the inherent material elasticity of flexible membrane 80 provides tensile forces that pull central hub 96 into abutment with bore 74 via spokes 94. Flexible membrane 80 is moved from the sealing position to the dispensing position by insertion of the syringe nose 108 through bore 74. Insertion of syringe nose 108, as shown in FIG. 5, causes central hub 96 to be pushed away from bore 74, thereby breaking the seal therebetween. When central hub 96 is pushed away from bore 74, medicament 5 is able to flow from bottle storage cavity 30, through pores 98, and into syringe 100. Note that horizontal flange 86 acts to keep flexible membrane 80 in position relative to bottle 20 during the insertion of syringe nose 108. Upon withdrawal of the syringe 100, flexible membrane 80 returns to the sealing position.

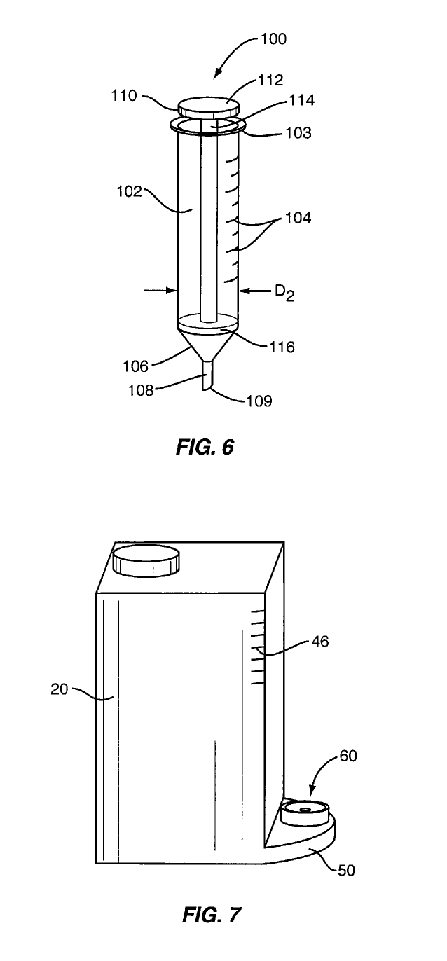

Referring to FIG. 6, the syringe 100 is a needleless syringe having a barrel 102 and a moveable plunger 110, as is conventional. The upper portion of barrel 102 is advantageously generally cylindrical with diameter D₂, with outwardly extending flange 103, and includes suitable graduation markings 104 to indicate the volume of liquid contained in barrel 102. The lower portion of barrel 102 includes a tapering section 106 that leads to hollow nose or cannula 108. The tip 109 of nose 108 is advantageously angled so that a portion may press against central hub 96, while allowing a gap to be formed therebetween. See FIG. 5. The plunger 110 is moveably coupled to barrel 102, and includes an upper flange 112, a shaft 114, and a suitable sealing tip 116. Movement of the plunger 110 causes material to be pulled into or expelled from syringe 100 via nose 108 in a convention fashion.

To use the medicine bottle 20 assembly, liquid medicament 5 is added to bottle 20, via opening 26, either by a user or at a factory or pharmacy. During this filling, the valve 70 is advantageously closed. Cap 28 is then secured in place. Assuming that syringe 100 is not connected to bottle 20, syringe 100 is coupled to bottle 20 by snap-fitting syringe 100 into recess 42. This snap fit connection may be achieved by having the entry to recess be sized slightly smaller than recess 42. For example, recess 42 may be an partial-cylindrical channel with a cross-sectional diameter of D₁ about its longitudinal axis 44, with D₁ advantageously the same as D₂ or just slightly larger than D₂. Recess 42 may have an angular sweep of slightly greater than 180°, such that the width W of the lateral entry to recess 42 is slightly less than both D₁ and D₂. See FIG. 2. Of course, recess 42 need not be partial-cylindrical, and other cross-sectional shapes, longitudinally uniform or otherwise, may be used if desired. As can be appreciated, the cross-sectional shape of recess advantageously mimics that of syringe barrel 102. Advantageously, the material of bottle 20 proximate recess 42 is able to deflect slightly, so that the syringe barrel 102 is received into recess 42 in a snap-fit fashion. However, the grip of bottle 20 on syringe 100 is advantageously not so tight as to prevent movement (vertical in FIG. 1) of syringe 100 along bore axis 76. As such, when it is desired to dispense a measured portion of medicament 5, syringe 100 may be pushed down toward outlet port 60, so that nose 108 enters bore 74 of annular insert 72, and displaces flexible membrane 80 to thereby automatically opens valve 70. Advantageously, flange 103 on syringe 100 abuts against bottle top 24, or some other suitable stop, so as to prevent over-insertion of syringe 100. See FIG. 1. Additionally and/or alternatively, the hub 96 may abut against bottle 20 to aid in preventing over-insertion, as shown in FIG. 5. With syringe 100 inserted, the plunger 110 on syringe 100 is pulled upward to cause the medicament 5 to flow into syringe 100 from cavity 30 via pores 98 and outlet port 60. Advantageously, the syringe's gradations 104 are visible during this filling process, so that the user may easily determine the amount of medicament 5 in the syringe 100 without having to remove the syringe 100. During the syringe filling process, downward pressure on the syringe barrel 102 helps steady bottle 20. In addition, the fluid pressure differential between the static “head” pressure of the fluid in the cavity 30 and the lack thereof in the syringe 100 helps move the medicament 5 to the syringe 100 during the syringe 100 filling process. Indeed, nothing but the static head pressure of the medicament 5 in cavity 30 is required to supply the medicament 5 to outlet port 60 when valve 70 is open. Once the desired amount of medicament 5 is dispensed, the syringe 100 may be decoupled from the bottle 20, and the medicament 5 dispensed from the syringe 100 in a conventional fashion. Note that removal of the syringe 100 allows flexible membrane 80 to return to the sealing position, thereby automatically closing valve 70. After dispensing the medicament 5 from the syringe 100, the syringe 100 may optionally be cleaned and the re-coupled to the bottle 20 by snapping the syringe 100 into recess 42. Thus, the recess 42 may be used to both store the syringe 100 and to support the syringe 100 during the syringe filling process.

The discussion above has assumed that the outlet port 60 is disposed on a flange 50 that extends outward from the lower portion of bottle 20, but that still lies substantially within the footprint of the bottle 20. This arrangement facilitates the use of the recess 42 for support and storage of the syringe 100. However, such an arrangement is not required in all embodiments. For example, FIG. 7 shows another bottle 20 embodiment that includes an outwardly extending flange 50 (with outlet port 60) that lies substantially outside the footprint of the bottle 20. For such an embodiment, the bottle 20 may or may not include a storage recess 42 for syringe 100, which need not be aligned with outlet port 60.

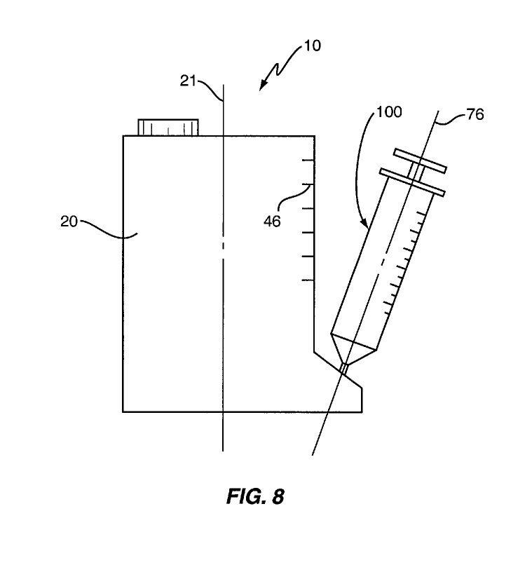

Further, the discussion above has assumed that outlet port 60 is oriented vertically, and this is believed advantageous. However, in some embodiments, the outlet port 60 may be oriented at an angle to vertical. For example, the outlet port 60 may be oriented outward at a 15° angle so that the syringe 100 may be inserted into the outlet port 60 at an angle relative to the bottle's longitudinal axis 21 (which is advantageously vertical). See FIG. 8. Also, in some embodiments, a removable cap (not shown) may be provided for selectively covering the outlet port 60. The removable cap may, if desired, be tethered to, or formed as part of, the flexible membrane 80 or other portion of the device 10.

In some embodiments, the bottle 20 may include optional graduation markings 46 proximate recess 42, as shown in FIG. 1, which may be used to indicate the amount of medicament 5 in an corresponding syringe 100 during filling thereof, or may be used to indicate the amount of medicament 5 remaining in cavity 30.

While the embodiments above have been discussed in terms of a valve 70 that automatically opens in response to the insertion of the syringe 100, this is not required in all embodiments. Indeed, a common twist handle flow valve 70 (not shown) may be employed, if desired, to control the flow of medicament 5 to outlet port 60. However, an automatically opening and closing valve 70 arrangement is believed most advantageous due to its simple operation.

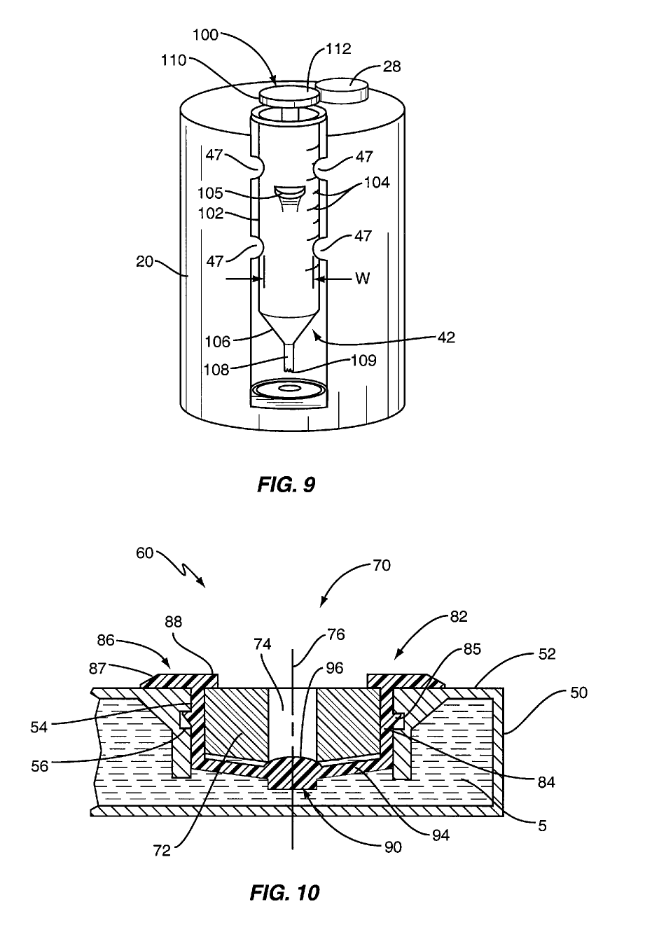

In some embodiments, the lateral entry into recess 42, in some embodiments, may not be of a uniform width, but may instead include a plurality of tabs 47 that act to narrow the entry to recess in selected locations. One embodiment of such and embodiment is shown in FIG. 9, with the size of the tabs 47 exaggerated for illustrative purposes. Alternatively, recess 42 may have a U-shaped cross-section with a width of just slightly more than D₂, with similar inwardly extending tabs disposed just inside of the entry to recess 42 so as to effectively narrow the entry of recess 42 to be W. And, as shown in FIG. 9, syringe 100 may, in some embodiments, include a laterally extending protrusion 105 on barrel 102. Such a protrusion 105 provides an additional means for the user to apply downward (and optionally upward) pressure on the syringe 100 so as to facilitate the syringe filling process.

Further, in some embodiments, the hub 96 on flexible membrane 80 may have an rounded upper surface to enhance sealing against bore 74. See FIG. 10. When such a configuration is used, it may be advantageous for the syringe nose 108 to have a fluted and/or scalloped tip portion 109 so as to provide suitable clearance for the inflow of medicament 5 during the syringe filling process.

The present invention may be carried out in other specific ways than those herein set forth without departing from the scope and essential characteristics of the invention. Further, the various aspects of the disclosed device and method may be used alone or in any combination, as is desired. The disclosed embodiments are, therefore, to be considered in all respects as illustrative and not restrictive, and all changes coming within the meaning and equivalency range of the appended claims are intended to be embraced therein.

What is claimed is:

1. A medicine bottle assembly, comprising:
a medicine bottle having an interior storage cavity and an outlet port, said outlet port disposed on an exterior of said bottle and in fluid communication with said storage cavity;
liquid medicament disposed in said storage cavity and defining a current fluid level; and
said outlet port disposed entirely below said current fluid level and oriented generally upward; said outlet port operative to selectively allow flow of said medicament out of said bottle.

2. The medicine bottle assembly of claim 1 further comprising a valve disposed proximate said outlet port and operative to control flow of said medicament through said outlet port.

3. The medicine bottle assembly of claim 2 wherein said valve is responsive to insertion of a hollow syringe tip thereinto to allow said medicament to flow out said outlet port.

4. The medicine bottle assembly of claim 3 further comprising a needless syringe having an angled tip.

5. The medicine bottle assembly of claim 2 wherein said valve comprises an elastically deformable element.

6. The medicine bottle assembly of claim 2 wherein said valve comprises an annular insert disposed in said outlet port and having a bore; said valve further comprising a flexible membrane movable between a sealing position sealing off said bore from fluid communication with said storage cavity and a dispensing position spaced from said bore so as to allow fluid to flow from said storage cavity to said bore.

7. The medicine bottle assembly of claim 6 wherein said flexible membrane comprises a solid section and a plurality of pores, said solid section abutting said bore when said flexible membrane is in said sealing position.

8. The medicine bottle assembly of claim 1 wherein said bottle exterior comprises a recess disposed above said outlet port, the recess adapted to receive and retain a needless syringe.

9. The medicine bottle assembly of claim 8 wherein said recess is aligned with said outlet port.

10. The medicine bottle assembly of claim 1 wherein said outlet port is oriented generally perpendicular to said fluid level.

11. The medicine bottle assembly of claim 1 wherein pressure in said storage cavity is ambient atmospheric pressure.

12. The medicine bottle assembly of claim 1 wherein said bottle comprises an upper opening for filling the storage cavity and a removable cap for closing said upper opening.

13. The medicine bottle assembly of claim 1 wherein said bottle is made from a polymer.

14. The medicine bottle assembly of claim 1 wherein said bottle is generally cylindrical.

15. The medicine bottle assembly of claim 1:
further comprising a needless syringe having a tip;
further comprising a valve disposed proximate said outlet port and operative to control flow of said medicament through said outlet port; said valve responsive to insertion of said syringe tip thereinto to allow said medicament to flow out said outlet port; said valve comprising an annular insert disposed in said outlet port and having a bore; said valve further comprising an elastic membrane movable between a sealing position sealing off said bore from fluid communication with said storage cavity and a dispensing position spaced from said bore so as to allow fluid to flow from said storage cavity to said bore;
wherein said outlet port is oriented generally perpendicular to said fluid level;
wherein said bottle exterior comprises a recess disposed above said outlet port, the recess aligned with said outlet port and adapted to receive and retain said syringe.

16. A method of dispensing medicine from a container bottle, comprising:
inserting a tip of a needless syringe into an outlet port from above; said outlet port disposed at a level below a current fill level of medicament stored in said bottle and oriented generally vertically upward;
moving a portion of said medicament from said bottle into said syringe while said syringe is mated to said outlet port;
thereafter, decoupling said syringe from said outlet port; and
thereafter, expelling medicament from said syringe.

17. The method of claim 16 further comprising automatically opening a valve associated with said outlet port in response to said inserting said syringe into said outlet port.

18. The method claim 16 further comprising retaining said syringe in a recess formed on an exterior of said bottle during said moving an amount of medicament from said bottle into said syringe.

19. The method of claim 16 further comprising disposing said syringe so that graduation markings associated therewith are visible during said moving an amount of medicament from said bottle into said syringe.

20. The method of claim 16 further comprising thereafter retaining said syringe in a recess formed on an exterior of said bottle.

21. A medicine bottle assembly, comprising:
a medicine bottle having an interior storage cavity and an exterior surface;
liquid medicament disposed in said storage cavity;
a needless syringe removably coupled to said bottle;
said bottle exterior surface defining a recess sized and configured to receive said syringe; and
said syringe disposed in said recess and in operative fluid communication with said medicament in said storage cavity.

22. The medicine bottle assembly of claim 21 wherein said syringe comprises gradation markings, said gradation markings being visible while said syringe is in operative fluid communication with said medicament in said storage cavity.

23. The medicine bottle assembly of claim 21 wherein said syringe is movable between an attached position secured to said bottle and disposed in said recess, and a detached position detached from said bottle.

24. The medicine bottle assembly of claim 21 wherein said syringe has a barrel section with an outer diameter, and wherein said recess has an entry width slightly less than said diameter.

25. The medicine bottle assembly of claim 21 wherein said syringe removably couples to said bottle via a snap-fit connection.

26. A method of dispensing medicine from a container bottle, comprising:
providing a needless syringe;
providing a medicine bottle having an interior cavity storing liquid medicament therein;
inserting said syringe into a recess defined on an exterior surface of said bottle;
said recess disposed opposite a sidewall of said bottle from said bottle cavity and sized and configured to receive said syringe;
moving a portion of said medicament from said bottle cavity to said syringe while said syringe is disposed in said recess;
thereafter, decoupling said syringe from said recess; and
thereafter, expelling medicament from said syringe.

27. The method of claim 26 wherein said moving a portion of said medicament from said bottle cavity to said syringe comprises passing said portion of medicament through a generally vertically oriented outlet port associated with said bottle; said outlet port disposed below a current fill level of said medicament in said bottle.

28. The method of claim 27 further comprising automatically opening a valve associated with said outlet port in response to insertion of said syringe into said outlet port.

29. The method of claim 26 wherein said syringe includes graduation markings; and
wherein said moving a portion of said medicament from said bottle cavity to said syringe comprises moving said portion of said medicament from said bottle cavity to said syringe while said graduation markings are visible.

30. The method of claim 26 further comprising thereafter re-coupling said syringe to said bottle at said recess and retaining said syringe in said recess.
